(19) United States
(12) Patent Application Publication
(10) Pub. No.: US 2015/0258301 A1
(43) Pub. Date: Sep. 17, 2015

(54) SLEEP STATE MANAGEMENT BY SELECTING AND PRESENTING AUDIO CONTENT

(71) Applicants: Mehul Trivedi, San Francisco, CA (US); Vivek Agrawal, San Francisco, CA (US); Jason Donahue, San Francisco, CA (US); Cristian Filipov, San Francisco, CA (US); Dale Low, San Francisco, CA (US)

(72) Inventors: Mehul Trivedi, San Francisco, CA (US); Vivek Agrawal, San Francisco, CA (US); Jason Donahue, San Francisco, CA (US); Cristian Filipov, San Francisco, CA (US); Dale Low, San Francisco, CA (US)

(73) Assignee: AliphCom, San Francisco, CA (US)

(21) Appl. No.: 14/214,254

(22) Filed: Mar. 14, 2014

(51) Int. Cl. A61M 21/02; A61B 5/00

(52) U.S. Cl. CPC A61M 21/02; A61B 5/4815

(57) ABSTRACT

Techniques for managing sleep states by selecting and presenting audio content are described. Disclosed are techniques for receiving data representing a sleep state, selecting a portion of audio content from a plurality of portions of audio content as a function of the sleep state, and causing presentation of an audio signal comprising the portion of audio content at a speaker. Audio content may be selected based on sleep states, such as sleep preparation, being asleep or sleeping, wakefulness, and the like. Audio content may be selected to facilitate sleep onset, sleep continuity, sleep awakening, and the like. Audio content may include white noise, noise cancellation, stating a user's name, presenting another message such as a recommendation, the news, or a user's schedule, a song or piece of music, and the like, and may be stored as a file, generated dynamically, and the like.

### FIELD

Various embodiments relate generally to electrical and electronic hardware, computer software, human-computing interfaces, wired and wireless network communications, telecommunications, data processing, and computing devices. More specifically, disclosed are techniques for managing sleep states by selecting and presenting audio content.

### BACKGROUND

Achieving optimal sleep is desirable to many people. Conventional devices may present audio content manually selected by a user. For example, to facilitate sleep onset, a user may set a device to present relaxing music for a certain time period. However, presentation of the relaxing music may stop at the end of the time period, even if the user does not fall asleep during the time period. For example, to wake a user, a user may select audio content such as a happy song to be presented at the time an alarm is set. However, this audio content will be presented, even if the user is in a deep sleep at the time the alarm is triggered, and a more soothing song may be more suitable for waking him up.

Thus, what is needed is a solution for managing sleep states by selecting and presenting audio content without the limitations of conventional techniques.

### BRIEF DESCRIPTION OF THE DRAWINGS

Various embodiments or examples (“examples”) are disclosed in the following detailed description and the accompanying drawings:

FIG. 1 illustrates a device with a sleep state manager, according to some examples;

FIG. 2 illustrates a network of devices to be used with a sleep state manager, according to some examples;

FIG. 3 illustrates an application architecture for a sleep state manager, according to some examples;

FIG. 4 illustrates examples of sleep states and audio content, according to some examples;

FIG. 5 illustrates other examples of sleep states and audio content, according to some examples;

FIG. 6 illustrates a network of devices of a plurality of users, the devices to be used with sleep state managers, according to some examples;

FIG. 7 illustrates a process for a sleep state manager, according to some examples;

FIG. 8 illustrates another process for a sleep state manager, according to some examples;

FIG. 9 illustrates another process for a sleep state manager, according to some examples; and

FIG. 10 illustrates a computer system suitable for use with a sleep state manager, according to some examples.

### DETAILED DESCRIPTION

Various embodiments or examples may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

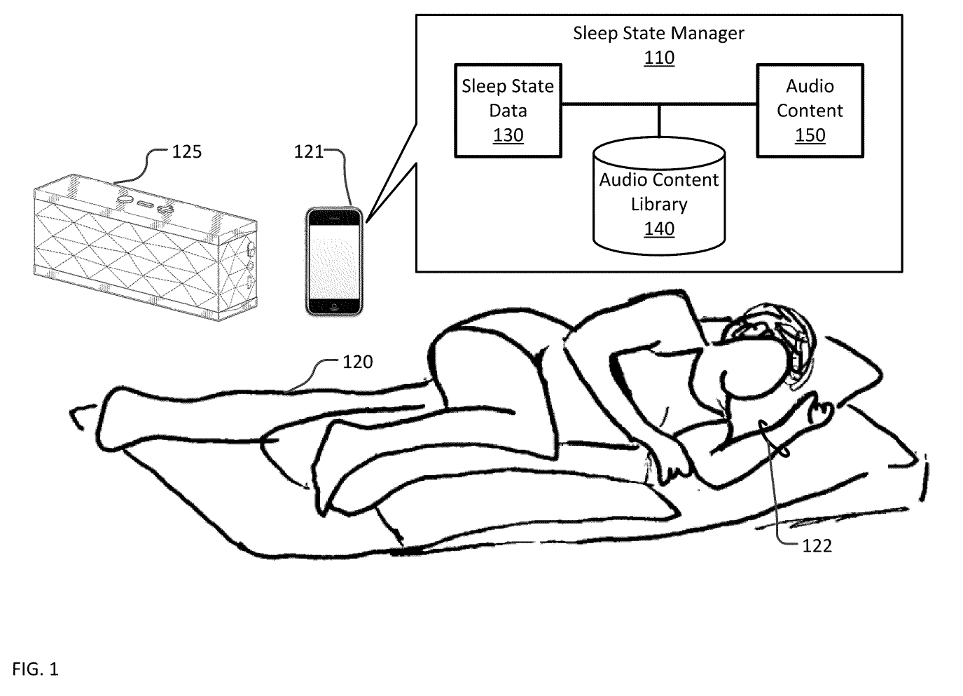

FIG. 1 illustrates a device with a sleep state manager, according to some examples. As shown, FIG. 1 includes sleep state manager 110, data representing a sleep state 130, audio content library 140, audio content 150, user 120, smartphone 121, data-capable strapband or band 122, and speaker box or media device 125. Sleep state manager 110 may be configured to select and present audio content 150 at media device 125 to facilitate, encourage, or help achieve a continuity or transition of sleep states or sleep stages of user 120 based on sensor data received from one or more sensors coupled to smartphone 121, band 122, media device 125, or another wearable device or device. In some examples, sleep state manager 110 may receive data representing a sleep state or sleep stage 130. A sleep state may be a period or step in the process of sleep. Sleep may proceed in cycles of sleep states, wherein one or more sleep states are repeated during the process of sleep. A sleep state may further be classified or broken down into sub-sleep states. A sleep state may be, for example, sleep preparation, sleeping or being asleep, and wakefulness. Sleep preparation may be an activity or time during which user 120 prepares to sleep. Sleep preparation may include, for example, entering a bedroom, brushing one's teeth, getting into bed, and the like. Sleeping may be a state characterized by altered consciousness, such as a relatively inhibited sensory activity, relatively inhibited movement of voluntary muscles, and the like. The state of sleeping or being asleep, for example, may further be classified into the sub-sleep states of light sleep or deep sleep. Deep sleep, for example, may include rapid eye movement (REM) sleep, which may be a stage of sleep characterized by rapid and random movement of the eyes. Wakefulness may be a state in which user 120 is conscious after user 120 is woken up from being asleep.

Sleep state manager 110 may be configured to select a portion or piece of audio content 150 from a plurality of portions or pieces of audio content stored in an audio content library 140 as a function of sleep state data 130. Selected audio content 150 may be stored in audio content library 140 and may be retrieved from audio content library 140. Sleep state manager 110 may determine that audio content 150 may be used to help user 120 continue or maintain his current sleep state. Sleep state manager 110 may select audio content 150, such as white noise, to help mask or obsure background, interfering, or unwanted noise, to help user 120 remain asleep. White noise may cover up unwanted sound by using auditory masking White noise may reduce or eliminate awareness of pre-existing sounds in a given area. White noise may be used to affect the perception of sound by using another sound. In some examples, white noise may be an audio signal whose amplitude is constant throughout the audible frequency range. White noise may be an audio signal having random frequencies across all frequencies or a range of frequencies. For example, white noise may be a blend of high and low frequencies. In other examples, white noise may be an audio signal with minimal amplitude and frequency fluctuations, such as nature sounds (e.g., rain, ocean waves, crickets chirping, and the like), fan or machine noise, and the like. Sleep state manager 110 may select audio content 150 to substantially cancel or attenuate background noise received at user 120. Background noise received at user 120 may be substantially canceled by, for example, providing an audio signal with that is a phase-shift or inverse of the background noise. If the interference is caused by, for example, the snoring of a sleeping partner of user 120, sleep state manager 110 may select audio content 150 to state the name of the sleeping partner. Audio content 150 may further suggest the sleeping partner to roll over. Audio content 150 may help stop or reduce the sleeping partner's snoring, and help user 120 remain asleep. Sleep state manager 110 may determine that audio content 150 may be used to help user 120 transition from his current sleep state to the next sleep state. Sleep state manager 110 may present white noise to help user 120 transition from sleep preparation to being asleep. If user 120 does not fall asleep within a time period (e.g., 30 minutes), for example, sleep state manager 110 may select audio content 150 to provide or state a recommendation to user 120. The recommendation may be, for example, to count backwards from 100, to get out of bed and do an exercise, and the like. Sleep state manager 110 may select audio content 150 that helps user 120 transition between sleep states quickly. Sleep state manager 110 may select audio content 150 that helps user 120 transition between sleep states gradually, which may be more comfortable or desirable for user 120, for example, because he is not suddenly woken from deep sleep or REM sleep. If user 120 is in deep sleep within a certain time period before an alarm is set to be triggered, for example, sleep state manager 110 may select audio content 150 to provide music at a low volume, to help user 120 transition to light sleep. Audio content 150 may be an identifier, name, or type of content to be presented as an audio signal. In some examples, audio content 150 may correspond to a file having data representing audio content stored in a memory. In other examples, audio content 150 may not correspond to an existing file and may be generated dynamically or on the fly. For example, audio content 150 may be configured to cancel a background noise. The background noise may be detected by a sensor coupled to sleep state manager 110, and audio content 150 may be generated dynamically to substantially cancel the background noise for user 120. Still, other audio content may be used.

Sleep state manager 110 may cause presentation of an audio signal having audio content 150 to be presented at a speaker, such as, media device 125. The audio signal may also be presented at two or more speakers. In some examples, sleep state manager 110 may present visual content or other signals at a screen, monitor, or other user interface based on the sleep state. In some examples, sleep state manager 110 may further be in data communication with other devices that may be used to adjust other environmental factors to manage a sleep state, such as dimming a light, shutting a curtain, raising a temperature, and the like. For example, to help user 120 transition from sleep preparation to falling asleep, sleep state manager 110 may present white noise at media device 125 and turn off the lights in the room. Sleep state manager 110 may be implemented at mobile device 121, or another device (e.g., media device 125, band 122, server (not shown), etc.).

Sleep state data 130 may be determined based on sensor data received from one or more sensors coupled to smartphone 121, band 122, media device 125, or another wearable device or device. A wearable device may be may be worn on or around an arm, leg, ear, or other bodily appendage or feature, or may be portable in a user's hand, pocket, bag or other carrying case. As an example, a wearable device may be band 122, smartphone 121, media device 125, a headset (not shown), and the like. Other wearable devices such as a watch, data-capable eyewear, cell phone, tablet, laptop or other computing device may be used. A sensor may be internal to a device (e.g., a sensor may be integrated with, manufactured with, physically coupled to the device, or the like) or external to a device (e.g., a sensor physically coupled to band 122 may be external to smartphone 121, or the like). A sensor external to a device may be in data communication with the device, directly or indirectly, through wired or wireless connection. Various sensors may be used to capture various sensor data, including physiological data, activity or motion data, location data, environmental data, and the like. Physiological data may include, for example, heart rate, body temperature, bioimpedance, galvanic skin response (GSR), blood pressure, and the like. Activity data may include, for example, acceleration, velocity, direction, and the like, and may be detected by an accelerometer, gyroscope, or other motion sensor. Location data may include, for example, a longitude-latitude coordinate of a location, whether user 120 is in or within a proximity of a building, room, or other place of interest, and the like. Environmental data may include, for example, ambient temperature, lighting, background noise, sound data, and the like. Sensor data may be processed to determine a sleep state of user 120. For example, when one or more sensors detect a low lighting, a low activity level, and a location in a bedroom, user 120 may be preparing to sleep (e.g., in the sleep preparation state). As another example, when one or more sensors detect that user 120 has not moved for a time period, user 120 may be in deep sleep. This evaluation of sensor data may be done internally by sleep state manager 110 or externally by another device in data communication with sleep state manager 110. In some examples, sensor data is evaluated by a remote device, and data representing a sleep state 130 is transmitted from the remote device to sleep state manager 110. In some examples, sleep state manager 110 may be implemented or installed on smartphone 121 (as shown), or on band 122, media device 125, a server (not shown), or another device, or may be distributed on smartphone 121, band 122, media device 125, a server, and/or another device. Still, other implementations of sleep state manager 110 are possible.

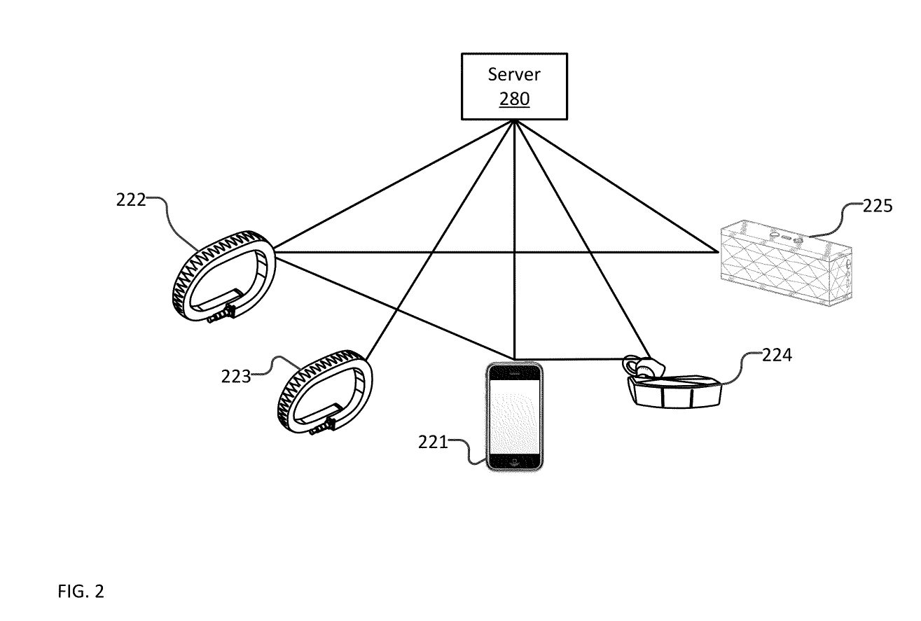

FIG. 2 illustrates a network of devices to be used with a sleep state manager, according to some examples. As shown, FIG. 2 includes smartphone 221, data-capable bands 222-223, headset 224, speaker box or media device 225, and server or node 280. Node 280 may be a server, or another device having a memory accessible by a plurality of users (e.g., another wearable device, or another computing device). Server 280 may be a computer or computer program configured to provide a network service or a centralized resource to devices 221-225. Server 280 may have a memory accessible by devices 221-225. As shown, devices 221-225 may be in direct data communication (e.g., directly communicating with each other) or indirect data communication (e.g., communicating with server 280, which then communicates with another device). Other devices, such as a computer, laptop, watch, and the like, may be used. One or more devices 221-225 of a user may be used by or with a sleep state manager. For example, a user may have band 222 worn on an arm, band 223 worn on a leg, and smartphone 221 and media device 225 placed next to or close to her. One or more of devices 221-225 may be physically coupled to a sensor such as a sound sensor, a temperature sensor, a motion sensor, and the like. Devices 221-225 may also be in data communication with one or more remote sensors. Sensor data from devices 221-225 may be used in conjunction to a sleep state of a user. Sensor data may be transmitted from devices 221-225 to a sleep state manager, directly or indirectly (e.g., through a node), using wired or wireless communications. The sleep state manager may be executed on smartphone 221, a computing device (e.g., devices 221-225, or others), server 280, or distributed over server 280 and/or one or more computing devices. Devices 221-225 may also access server 280 for audio content and other applications or resources. In some examples, sensor data may be received at band 223 and transmitted to server 280 for evaluation. Data representing a sleep state may be determined at server 280 and transmitted to a sleep state manager, which may be implemented on smartphone 221 or media device 225 or another device. The sleep state manager may select audio content from an audio content library, which may be stored on a local memory, server 280, or another device. The sleep state manager may cause presentation of the audio content on media device 225. In other examples, headset 224 or another device may be used to present the audio content. For example, sleep state manager may cause presentation of white noise, a signal configured to cancel background noise, a recommendation, a user's name, and the like. Still, other implementations and/or network configurations may be used with a sleep state manager.

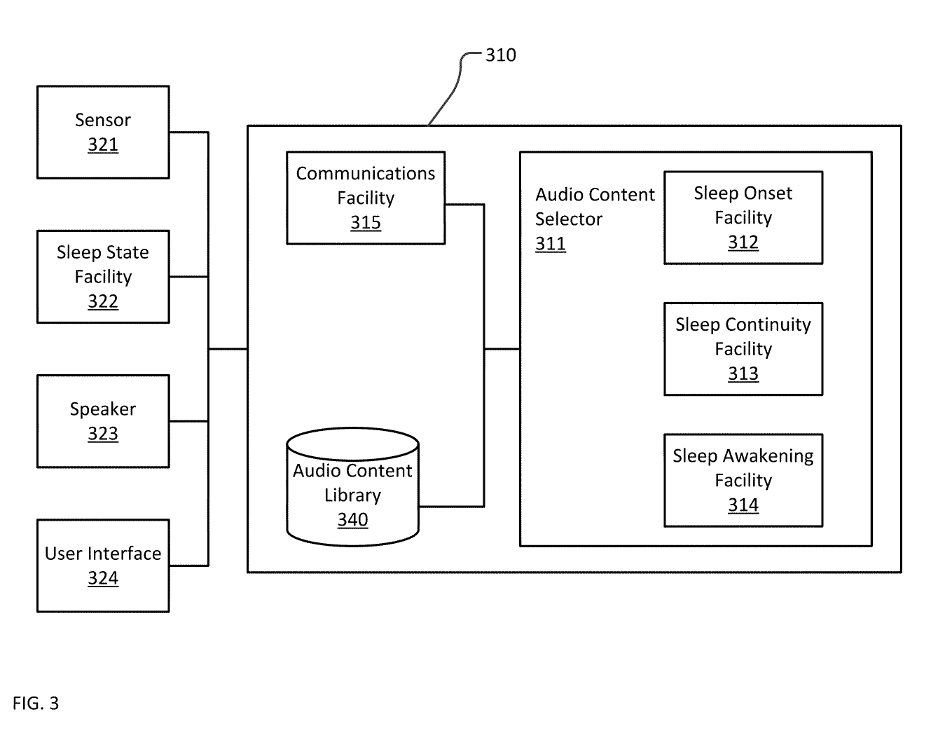

FIG. 3 illustrates an application architecture for a sleep state manager, according to some examples. As shown, FIG. 3 includes a sleep state manager 310, an audio content selector 311, a sleep onset facility 312, a sleep continuity facility 313, a sleep awakening facility 314, a communications facility 315, an audio content library 340, a sensor 321, a sleep state facility 322, a speaker 323, and a user interface 324. As used herein, “facility” refers to any, some, or all of the features and structures that are used to implement a given set of functions, according to some embodiments. Elements 311-315 and 340 may be integrated with or installed on sleep state manager 310 (as shown), or may be remote from and in data communication with sleep state manager 310 through communications facility 315, using wired or wireless communication. Elements 321-324 may be implemented locally on or remotely from (as shown) to sleep state manager 310. Audio content library 340 may be stored or implemented on a memory or data storage that is local to sleep state manager 310 (as shown) or external to sleep state manager 310 (e.g., stored on a server or other external memory). For example, audio content library 340 may be implemented using various types of data storage technologies and standards, including, without limitation, read-only memory (“ROM”), random access memory (“RAM”), dynamic random access memory (“DRAM”), static random access memory (“SRAM”), static/dynamic random access memory (“SDRAM”), magnetic random access memory (“MRAM”), solid state, two and three-dimensional memories, Flash®, and others. Audio content library 340 may also be implemented on a memory having one or more partitions that are configured for multiple types of data storage technologies to allow for non-modifiable (i.e., by a user) software to be installed (e.g., firmware installed on ROM) while also providing for storage of captured data and applications using, for example, RAM. Audio content library 340 may be implemented on a memory such as a server or node that may be accessible to a plurality of users, such that one or more users may share, access, create, modify, or use audio content. Once captured and/or stored in audio content library 340, data may be subjected to various operations performed by other elements of sleep state manager 310, as described herein.

In some examples, communications facility 315 may receive data representing a sleep state from sleep state facility 322. In other examples, sleep state facility 322 may be implemented locally on sleep state manager 310. Sleep state facility 322 may be configured to process sensor data received from sensor 321 and determine a sleep state. Sleep state facility 322 may be coupled to a memory storing one or more sensor data patterns or criteria indicating various sleep states. For example, a sensor data pattern having low lighting, low activity level, and location in a bedroom, may be used to determine a sleep state of sleep preparation. As another example, bioimpendance, galvanic skin response (GSR), or other sensor data may be used to determine light sleep or deep sleep. As another example, high activity level after a state of sleeping may be used to determine a sleep state of wakefulness. Sleep state facility 322 may compare sensor data to one or more sensor data patterns to determine a match, or a match within a tolerance, and determine a sleep state. Sleep state facility 322 may generate a data signal representing the sleep state.

Audio content selector 311 may be configured to select a portion of audio content from a plurality of portions of audio content stored in audio content library 340 based on the sleep state determined by sleep state facility 322. As described above, audio content may be an identifier, name, or type of content to be presented as an audio signal. In some examples, audio content may correspond to a file having data representing audio content, and the file may be stored in audio content library 340 or another memory. In other examples, audio content may correspond to an audio signal that is to be generated dynamically or on the fly. For example, audio content may include white noise, which may include an audio signal having a constant amplitude over random frequencies. In one example, the random frequencies may be generated dynamically (e.g., based on a random number generator). In another example, the white noise may be a sound recording, which may be looped or presented repeatedly. Audio content may be preinstalled or pre-packaged in audio content library 340, or may be entered or modified by the user. For example, audio content library 340 may be preinstalled with a white noise signal using random frequencies over all frequencies. A user may add another white noise to audio content library 340 that includes a signal using random frequencies over lower frequencies only. A user may also add a music or song to audio content library 340, by adding an identifier of the music (which may be used to retrieve a file having data representing the music from another memory, a server, or over a network, and the like), or by adding and storing a file having data representing the music on audio content library 340. Audio content to be used for a certain sleep state may be set by default (e.g., preinstalled, integrated with firmware, etc.) or may be entered or modified by the user. For example, by default, sleep state manager 310 may select white noise to be presented during sleep preparation. For example, a user may modify the audio content selection, such that, a song is presented during sleep preparation. For example, a user may instruct sleep state manager 310 to select a song during a certain time period of sleep preparation (e.g., during the first 10 minutes of sleep preparation), and if he is not yet asleep, select white noise for the remainder of sleep preparation.

Audio content selector 311 may include modules or components such as sleep onset facility 312, sleep continuity facility 313, and sleep awakening facility 314. Sleep onset facility 312 may be configured to select audio content to help or facilitate sleep onset. Sleep onset may be a transitioning from sleep preparation to being asleep. In one example, communications facility 315 may receive data representing the sleep state of sleep preparation. Sleep onset facility 312 may select white noise, music, or other audio content from audio content library 340 to help the user fall asleep. In some examples, sleep onset facility 312 may determine that the user has been in sleep preparation state for over a certain time period (e.g., 30 minutes), and still has not fallen asleep. Sleep onset facility 312 may select to present a recommendation at a speaker and/or other user interface. One recommendation may be configured to relax a user's mind, such as counting backwards from 100, breathing slowly, or the like. Another recommendation may be configured to decrease a user's physical energy, such as doing an exercise, taking a walk, or the like. Other recommendations may be used. In some examples, sleep onset facility 312 may provide a series of recommendations to the user at speaker 323. A first recommendation may be, for example, to walk from the bedroom to the hallway, and a second recommendation may be, for example, to stretch the user's hip to the right. In some examples, speaker 323 may be portable. In some examples, the user may take speaker 323 out of the bedroom and into the hallway. Moving speaker 323 away from the bedroom may help reduce the interference or disturbance that the audio content is causing to the user's sleeping partner. After presenting a recommendation, sleep onset facility 312 may select another audio content to facilitate sleep onset. Sleep onset facility 312 may stop (e.g., abruptly or gradually) presenting the audio content after receiving data representing a sleep state of being asleep.

Sleep continuity facility 313 may be configured to select audio content to help or facility sleep continuity. Sleep continuity may be remaining in a sleeping state, a light sleep state, or a deep sleep state. Sleep continuity may be returning to a sleeping state after being briefly in a wakefulness state, for example, returning to a sleeping state after being woken up by an interference (e.g., a dog bark, a siren, and the like). In some examples, sleep continuity facility 313 may receive data representing a sleep state of being asleep or sleeping. Sleep continuity facility 313 may also receive data representing an interference. An interference may be a sensory signal (e.g., audio, visual/light, temperature, etc.) that may interfere with or disturb sleep. Sensor 321 may capture a sensory signal, and an interference facility (not shown) may process the sensor data to determine an interference has occurred. For example, an interference facility may have a memory storing a set of patterns, criteria, or rules associated with interferences. For example, an audio signal above a threshold decibel (dB) level may indicate an interference. For example, a light above a threshold level may indicate an interference. Sleep continuity facility 313 may select audio content to help or facilitate sleep continuity despite the interference. For example, sleep continuity facility 313 may present white noise to mask an audio interference. In some examples, sleep continuity facility 313 may select audio content based on data representing a sleep state after the interference. For example, after data representing an interference is received, data representing deep sleep is received. Sleep continuity facility 313 may not to present audio content since the user remained in deep sleep. As another example, after data representing an interference is received, data representing light sleep is received. Sleep continuity facility 313 may select to present white noise. As another example, after data representing an interference is received, data representing wakefulness is received. Sleep continuity facility 313 may select to present a signal configured to cancel the background noise. Depending on the volume of an audio interference, sleep continuity facility 313 may also adjust the volume of the presentation of the audio content. In some examples, the interference may be caused by the snoring of the user's sleeping partner. In some examples, sleep continuity facility 313 may select to present white noise or a noise cancellation signal to mask or substantially cancel or attenuate the sound of snoring. In other examples, sleep continuity facility 313 may select to audio content stating the name of the user's sleeping partner. The audio content may also make a suggestion to the sleeping partner, for example, “Sam, please roll over.” A person's auditory senses may be more sensitive to her own name, and thus may be alert to or hear her name at a lower volume. A sleeping partner may be sensitive to audio content stating the sleeping partner's name, while the user may not be sensitive to or be alerted by the audio content.

Sleep awakening facility 314 may be configured to select audio content to help or facilitate waking up, or transitioning from sleeping to wakefulness. Data representing a sleep state, such as being asleep, being in deep sleep, and the like, may be received. Data representing a time at which to present an audio content may also be received. For example, a user may set an alarm clock for 8 a.m. using user interface 324. Sleep awakening facility 314 may select audio content as a function of a time period between a first time when the data representing a sleep state was received and a second time when the audio content is to be presented. For example, data representing being asleep may be received at 12 midnight, and the time to present the audio content may be set to 8 a.m. Sleep awakening facility 314 may select audio content based on the time the user was asleep, for example, 8 hours. Since the user may be well rested, sleep awakening facility 314 may select to present the daily news or a news story (e.g., reading off headlines) to wake the user up. Data representing the news may be received from a server or over a network using communications facility 315, or using other methods. Sleep awakening facility 314 may also select to present or read out the user's schedule to wake the user up. Data representing the user's schedule may be received from a server or over a network using communications facility 315, or may be stored in a memory local to sleep state manager 310. The user may enter his schedule into memory using user interface 324. As another example, data representing a sleep state may be received at regular intervals (e.g., every 15 minutes), and sleep awakening facility 314 may determine that the user was in deep sleep for only 1 hour. Since the user may not be well rested, sleep awakening facility 314 may select a piece of music (e.g., a relaxing song) to wake the user up. After audio content is selected by sleep awakening facility 314 and presented at speaker 323, data representing a sleep state, such as being asleep, may be received. If data representing being asleep is received after a time period (e.g., 10 minutes) after the audio content is presented at speaker 323, sleep awakening facility 314 may select another audio content, such as a loud alarm, to wake the user up.

Communications facility 315 may include a wireless radio, control circuit or logic, antenna, transceiver, receiver, transmitter, resistors, diodes, transistors, or other elements that are used to transmit and receive data, including broadcast data packets, from other devices. In some examples, communications facility 315 may be implemented to provide a “wired” data communication capability such as an analog or digital attachment, plug, jack, or the like to allow for data to be transferred. In other examples, communications facility 315 may be implemented to provide a wireless data communication capability to transmit digitally encoded data across one or more frequencies using various types of data communication protocols, such as Bluetooth, Wi-Fi, 3G, 4G, without limitation.

Sensor 321 may be various types of sensors and may be one or more sensors. Sensor 321 may be configured to detect or capture an input to be used by sleep state facility 322 and/or sleep state manager 310. For example, sensor 321 may detect an acceleration (and/or direction, velocity, etc.) of a motion over a period of time. In some examples, sensor 321 may include an accelerometer. An accelerometer may be used to capture data associated with motion detection along 1, 2, or 3-axes of measurement, without limitation to any specific type of specification of sensor. An accelerometer may also be implemented to measure various types of user motion and may be configured based on the type of sensor, firmware, software, hardware, or circuitry used. In some examples, sensor 321 may include a gyroscope, an inertial sensor, or other motion sensors. In other examples, sensor 321 may include an altimeter/barometer, light/infrared (“IR”) sensor, pulse/heart rate (“HR”) monitor, audio sensor (e.g., microphone, transducer, or others), pedometer, velocimeter, GPS receiver or other location sensor, thermometer, environmental sensor, bioimpedance sensor, galvanic skin response (GSR) sensor, or others. An altimeter/barometer may be used to measure environmental pressure, atmospheric or otherwise, and is not limited to any specification or type of pressure-reading device. An IR sensor may be used to measure light or photonic conditions. A heart rate monitor may be used to measure or detect a heart rate. An audio sensor may be used to record or capture sound. A pedometer may be used to measure various types of data associated with pedestrian-oriented activities such as running or walking A velocimeter may be used to measure velocity (e.g., speed and directional vectors) without limitation to any particular activity. A GPS receiver may be used to obtain coordinates of a geographic location using, for example, various types of signals transmitted by civilian and/or military satellite constellations in low, medium, or high earth orbit (e.g., “LEO,” “MEO,” or “GEO”). In some examples, differential GPS algorithms may also be implemented with a GPS receiver, which may be used to generate more precise or accurate coordinates. In other examples, a location sensor may be used to determine a location within a cellular or micro-cellular network, which may or may not use GPS or other satellite constellations. A thermometer may be used to measure user or ambient temperature. An environmental sensor may be used to measure environmental conditions, including ambient light, sound, temperature, etc. A bioimpedance sensor may be used to detect a bioimpedance, or an opposition or resistance to the flow of electric current through the tissue of a living organism. A GSR sensor may be used to detect a galvanic skin response, an electrodermal response, a skin conductance response, and the like. Still, other types and combinations of sensors may be used. Sensor data captured by sensor 321 may be used by sleep state facility 322 (which may be local or remote to sleep state manager 310) to determine a sleep state. For example, an activity level detected by sensor 321 below a threshold level may indicate that the user is asleep. Sensor data captured by sensor 321 may also be used to determine other data, such as data representing an interference. For example, an audio signal detected by sensor 321 at a certain frequency and amplitude may be used to determine an interference, such as snoring and the like. Sensor data captured by sensor 321 may also be used by sleep state manager 310 to select audio content. For example, the selection of audio content may be a function of data representing a sleep state and other data, such as other sensor data, data representing an interference, and the like. Still, other uses and purposes may be implemented.

Speaker 323 may include hardware and software, such as a transducer, configured to produce sound energy or audible signals in response to a data input, such as a file having data representing a media content. Speaker 323 may be coupled to a headset, a media device, or other device. Sleep state manager 310 may select audio content from audio content library 340 based on sensor data received from sensor 321, and may cause presentation of the audio content at speaker 323.

User interface 324 may be configured to exchange data between a device and a user. User interface 324 may include one or more input-and-output devices, such as a keyboard, mouse, audio input (e.g., speech-to-text device), display (e.g., LED, LCD, or other), monitor, cursor, touch-sensitive display or screen, and the like. Sleep state manager 310 may use user interface 324 to receive user-entered data, such as uploading of audio content, selection of audio content for a certain sleep state, entry of a time to present audio content (e.g., triggering of an alarm), and the like. Sleep state manager 310 may also use user interface 324 to present information associated with sensor data received from sensor 321, data representing a sleep state, the audio content selected by sleep state manager 310, and the like. For example, user interface 324 may display a video content associated with the audio content presented at speaker 323. For example, user interface 324 may display the time period between sleep preparation and being asleep, the total amount of time being in deep sleep, and the like. As another example, user interface 324 may use a vibration generator to generate a vibration associated with a portion or piece of audio content (e.g., audio content used to wake a user up). As another example, a user may use user interface 324 to enter biographical information, such as age, sex, and the like. Biographical information may be used by sleep state manager 310 to select, tailor, or customize audio content. Biographical information may also be used by sleep state facility 322 to process sensor data to determine a sleep state. Still, other implementations of user interface 324 may be used.

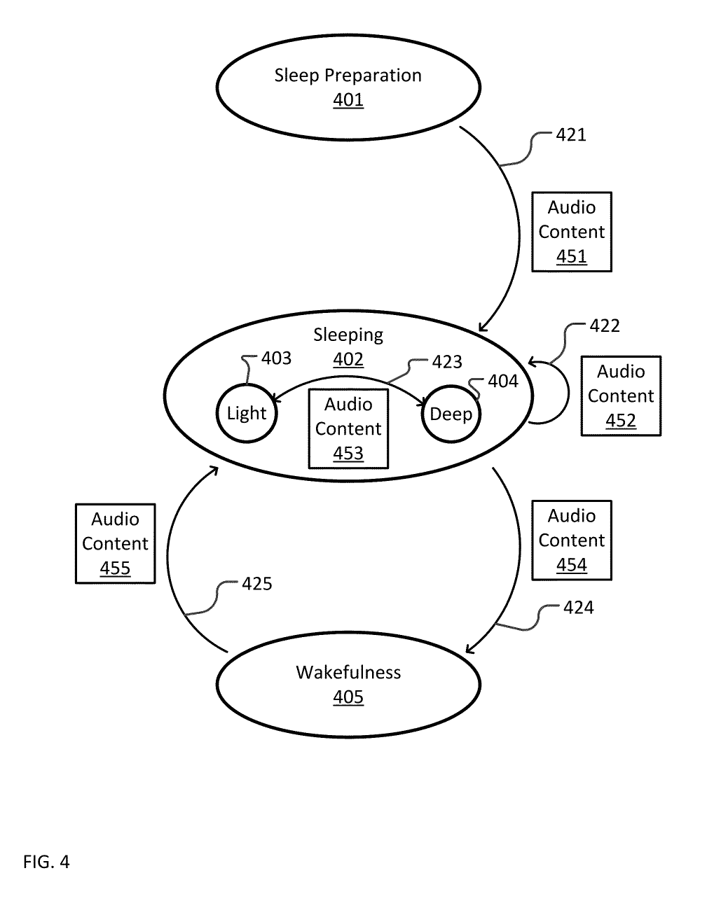

FIG. 4 illustrates examples of sleep states and audio content, according to some examples. As shown, FIG. 4 includes sleep states 401-405, sleep state transitions or continuations 421-425, and portions or pieces of audio content 451-455. Sleep states may be sleep preparation 401, sleeping or being asleep 402, light sleep 403, deep sleep 404, wakefulness 405, and the like. Sleep state transitions or continuations may be sleep onset 421, sleep continuity 422, transitioning between light sleep and deep sleep 423, waking up 424, and sleep continuity 425. Portions of audio content 421-425 may be selected as a function of sleep states 401-405. Portions of audio content 421-425 may also be selected as a function of sleep state transitions or continuations 421-425. In some examples, based on a sleep state being sleep preparation 401, audio content 451 may be selected and presented to facilitate sleep onset 421. In some examples, a sleep state may be sleeping 402. To maintain sleep continuity 422, audio content 452 may be selected. In some examples, an interference may be detected during sleeping 402, and audio content 452 may be selected to maintain sleep continuity 422. In some examples, data representing light sleep 403 or deep sleep 404 may be received, and audio content 453 may be selected to transition between them. In some examples, another audio content (not shown) may be selected to maintain continuity of light sleep 403 or deep sleep 404. A user may transition between light sleep 403 and deep sleep 404 multiple times while in the sleeping state 402. In some examples, a sleep state of wakefulness 405 may be detected, and audio content 455 may be selected to maintain sleep continuity 425. In some examples, a sleep state of sleeping 402 may be detected, and audio content 454 may be selected to facilitate waking up 424. In some examples, sleeping 402 may be detected after audio content 454 is presented, and another audio content (not shown) may be selected and presented.

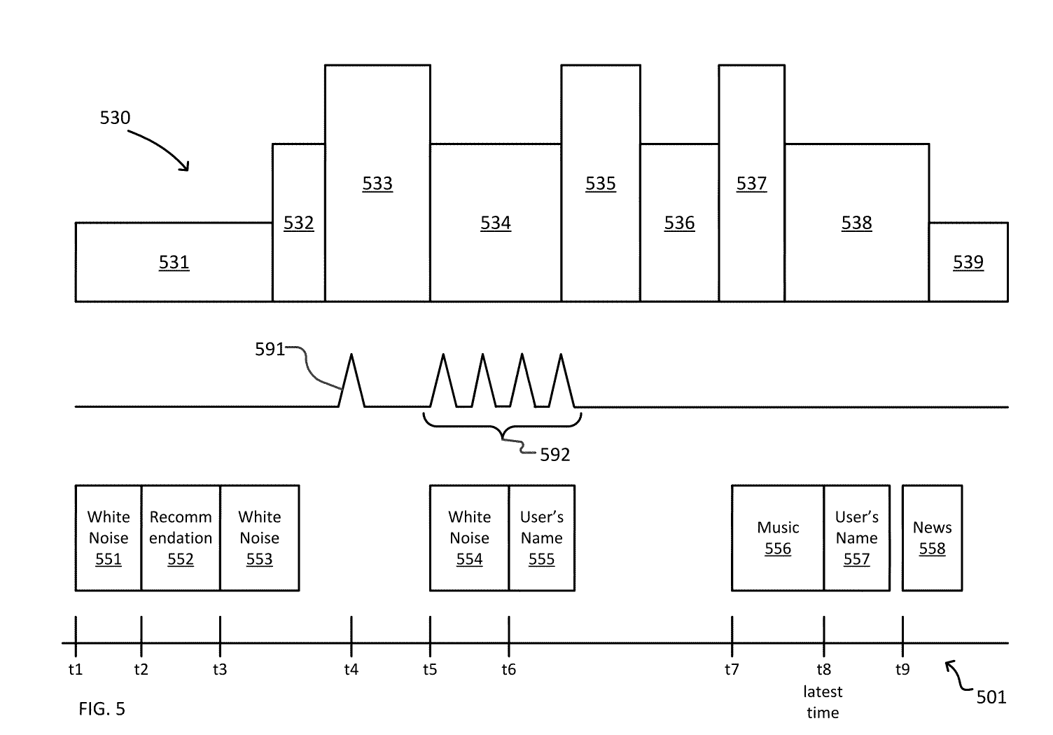

FIG. 5 illustrates other examples of sleep states and audio content, according to some examples. As shown, FIG. 5 includes a representation of sleep states 530, which may include states such as sleep preparation 531, light sleep 532, 534, 536, 538, deep sleep 533, 535, 537, wakefulness 539, and the like. FIG. 5 also includes a representation of interferences 591-592, portions of audio content 551-558, and timeline 501 having times t1-t9. In one example, at time t1, data representing sleep preparation 531 may be received, and white noise 551 may be selected and presented to facilitate sleep onset. After a certain time period, at time t2, data representing sleep preparation 531 may be received again, and recommendation 552 may be selected and presented. Recommendation 552 may suggest a relaxation exercise, a physical exercise, or the like to be performed by the user to facilitate sleep onset. After recommendation 552, at time t3, white noise 553 may be selected and presented again to facilitate sleep onset. In one example, data representing light sleep 532 and data representing deep sleep 533 may be received. During deep sleep 533, at time t4, interference 591 may be detected. As shown, for example, interference 591 may be a one-time, not repeated, or temporary disturbance, such as a dog bark, a siren, and the like. Data representing deep sleep 533 may continue to be received. Since the user was not disturbed or transitioned from deep sleep 533, no audio content may be presented. At time t5, another interference 592 is detected. As shown, for example, interference 592 may be a repeated or continuous disturbance, such as a sleeping partner's snoring. Data representing light sleep 534 may be received. Since the user was disturbed and transitioned from deep sleep 533 to light sleep 534, white noise 554 may be selected to mask interference 592 and facilitate sleep continuity. At time t6, data representing light sleep 534 may continue to be received. Audio content stating the name of the sleeping partner 555 may be selected and presented. Audio content 555 may further make a suggestion to the sleeping partner, such as rolling over. Audio content 555 may be presented at a low volume. A person's auditory senses may be more sensitive to hearing one's own name. An audio signal at a certain volume might not alert or disturb a person from sleep, but an audio signal at the same volume stating the person's name may be heard by the person while sleeping. Thus the sleeping partner may be alerted by audio content 555, while the user may not be disturbed by audio content 555. After stating the sleeping partner's name 555, interference 592 may stop. In one example, time t8 may be set to be a latest time at which the user is to wake up, for example, t8 may be a time for an alarm to be triggered. At time t7, data representing deep sleep 537 is received. To prepare or facilitate the waking up to occur at t8, at time t7, music 556 may be selected and presented. Music 556 may facilitate a transition from deep sleep 537 to light sleep 538. Music 556 may be presented at a low volume, and gradually increased in volume. At time t8, audio content 557 may be selected to wake the user up (e.g., an alarm may be triggered to wake the user up). A sleeping partner of the user, for example, may desire to be woken up at a later time (e.g., the sleeping partner set an alarm for a later time). The user may be more sensitive to hearing an audio signal of her name 557. Thus, the audio content stating the user's name 557 may be selected and presented at a low volume, which may facilitate the waking up of the user, while not disturbing the sleeping partner's sleep. In one example, after a certain time period after audio content 557 is presented, at time t9, data representing light sleep 538 may be received. This may indicate that the user was not woken up by audio content 557. Audio content 558 which may be louder or more disruptive, such as the news, may be selected. Data representing wakefulness 529 may then be received. Still, data representing other sleep states may be detected and received, other interferences may be detected and received, and other audio content may be selected and presented as a function of the sleep states.

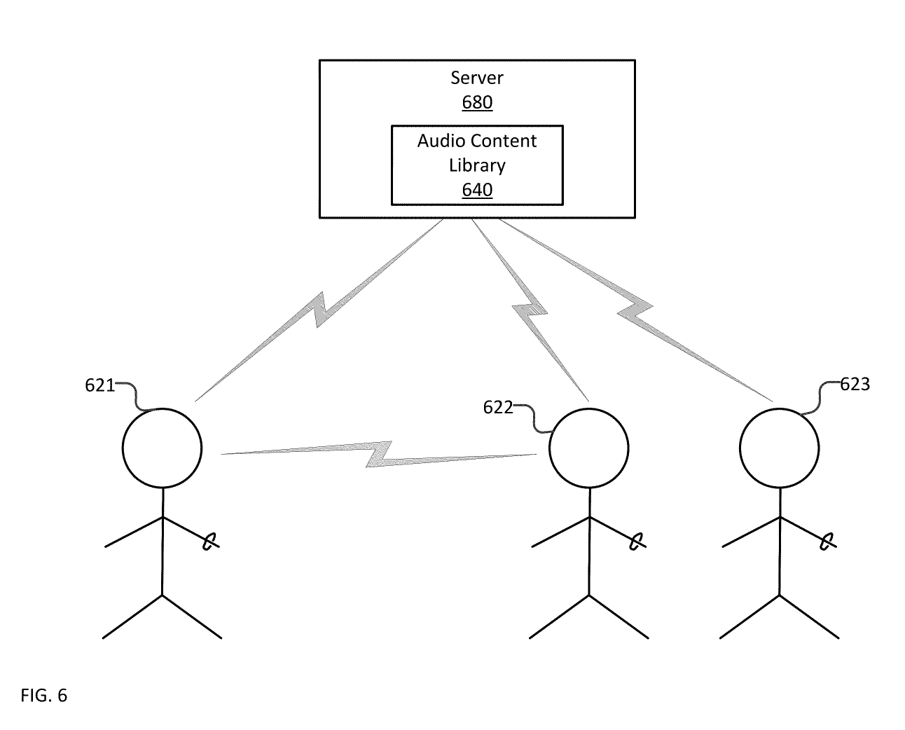

FIG. 6 illustrates a network of devices of a plurality of users, the devices to be used with sleep state managers, according to some examples. As shown, FIG. 6 includes server or node 680, audio content library 640, and users 621-623. Each user 621-623 may use one or more devices having a sleep state manager. The devices of users 621-623 may communicate with each other over a network, and may be in direct data communication with each other, or be in data communication with server 680. Server 680 may include audio content library 640. Audio content library 640 may store one or more portions of audio content. Users 621-623 may upload, share, or store audio content on audio content library 640, and may retrieve or download audio content from audio content library 640. For example, a portion of audio content may be good at facilitating sleep onset of user 621 (e.g., the time for sleep onset is short when this audio content is presented). This audio content may be uploaded to audio content library 640 and shared with users 622-623. This audio content may be automatically marked as “good” by a sleep state manager. As another example, audio content may include a piece of music marked as “favorite” by user 621. A device of user 622 may directly communicate with a device of user 621, and retrieve the music piece. Audio content may be downloaded, purchased, or retrieved from a marketplace. A marketplace may be a portal, website, or centralized service from which a plurality of users may retrieve or download resources, such as audio content. A marketplace may be accessible over a network, such as using server 680, the Internet, or other networks.

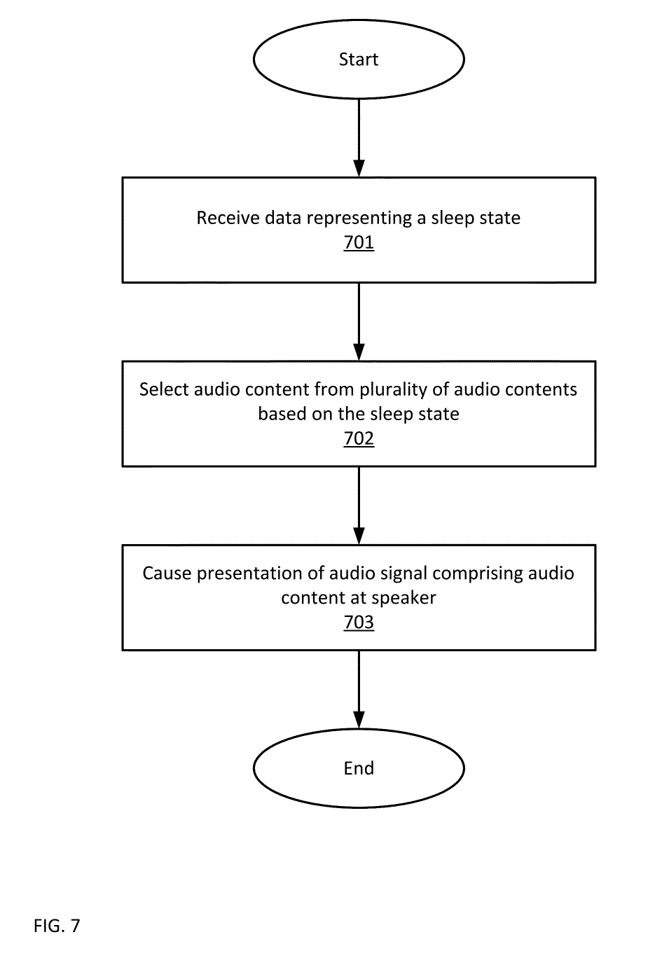

FIG. 7 illustrates a process for a sleep state manager, according to some examples. At 701, data representing a sleep state may be received. The sleep state may be determined based on sensor data received at one or more sensors. For example, sensor data may be compared to one or more data patterns, rules, or criteria to determine a sleep state. For example, certain criteria corresponding to various sleep states may be specified for sensor data, such as bioimpendance, activity level, lighting level, sound level, location, and the like. One or more sensors may be used, and the sensors may be local to or remote from the sleep state manager. At 702, a portion of audio content may be selected from a plurality of audio content based on the sleep state. The audio content may also be selected as a function of other data, such as data representing an interference or other sensor data. The audio content may be stored as a static file (e.g., a music file), or it may be dynamically created (e.g., a reading of the daily news is dynamically created as the daily news is received). The plurality of audio content may be stored in an audio content library, which may be local to or remote from the sleep state manager. The plurality of audio content may be stored on a memory that is accessible by a plurality of users. At 703, presentation of an audio signal comprising the audio content at a speaker may be caused. The speaker may be coupled to a media box, speaker box, headset, or other device. The speaker may be local to or remote from the sleep state manager. Still, other processes may be possible.

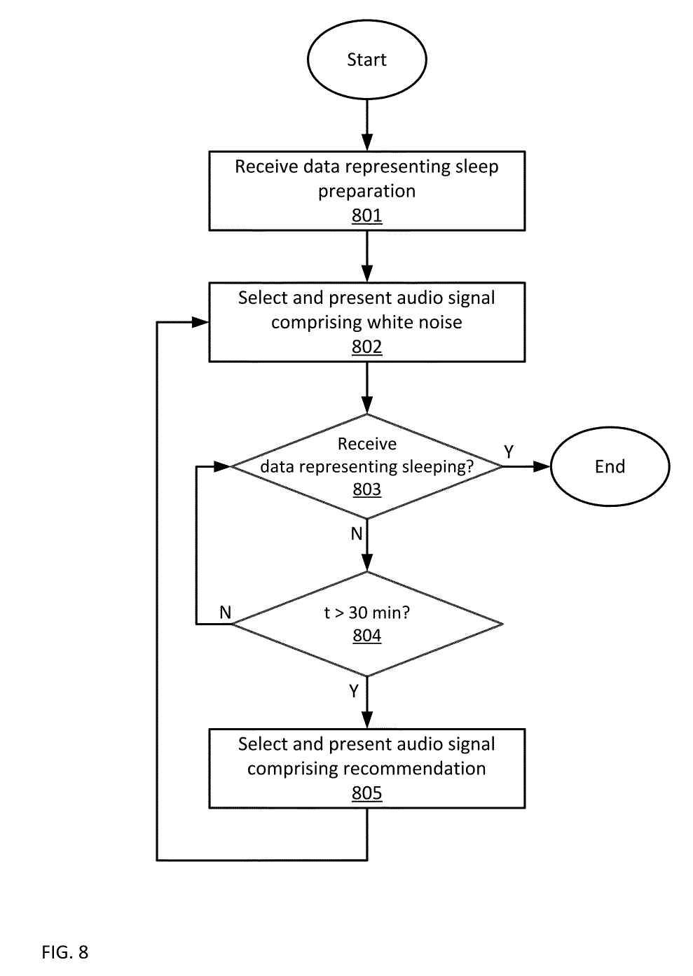

FIG. 8 illustrates another process for a sleep state manager, according to some examples. At 801, data representing a sleep state of sleep preparation may be received. At 802, a portion of audio content comprising white noise may be selected and presented. The audio signal comprising white noise may be selected to facilitate sleep onset. At 803, an inquiry may be made as to whether data representing a sleep state of sleeping is received. If yes, the process ends. Another process for maintaining sleep continuity or for facilitating waking up may proceed. If no, the process goes to 804, and an inquiry may be made as to whether the time since the data representing sleep preparation was received has exceeded a threshold, e.g., 30 minutes. If no, the process goes to 803, and an inquiry may be made as to whether data representing sleeping is received. The process may continue to wait for data representing sleeping to be received until the time has passed the threshold. If yes, then the process goes to 805. The time may have passed the threshold, and data representing sleep preparation may continue to be received. An audio signal comprising a recommendation may be selected and presented. The recommendation may suggest activities or actions that may facilitate sleep onset. The process goes back to 802, and an audio signal comprising white noise is selected and presented. The process may continue until data representing sleeping is received at 803. Still, other processes may be possible.

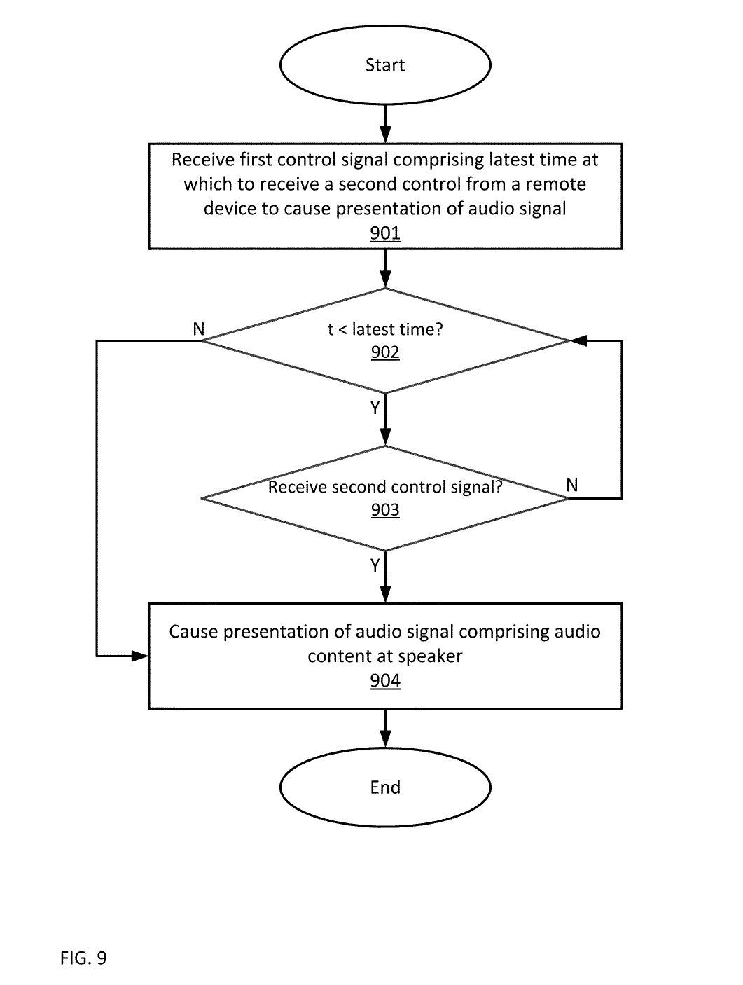

FIG. 9 illustrates another process for a sleep state manager, according to some examples. In some examples, a sleep state manager may have a fail-safe mode. A user may set a latest time at which audio content (e.g., an alarm) is to be presented in order to wake the user up. Sensor data may be captured and used to determine data representing a sleep state. Within a certain period before the latest time (e.g., 30 minutes before the latest time), if data representing a certain sleep state, such as light sleep, is received, then the audio content may be presented at this time. This may facilitate the waking up of the user, as the user may be woken during light sleep rather than deep sleep. If data representing light sleep is not received before the latest time, then the audio content may be presented at the latest time. For example, a user may set a time of 8 a.m. to be the latest time at which an alarm is to be triggered, and the alarm is to be triggered if and when light sleep is detected within a 30-minute period before 8 a.m., or the alarm is to be triggered at the latest time. In some examples, a first device may determine and generate the data representing a sleep state, and a second device may select and present audio content based on the data representing the sleep state. The first device and the second device may be in data communication with each other, and the second device may receive the data representing a sleep state from the first device. The data representing a sleep state may function as a control signal to the second device to present the audio content (e.g., trigger the alarm). In some examples, the second device may not receive data representing a sleep state due to an error or an unexpected event. The second device may not receive a control signal to trigger an alarm before the latest time set by the user. For example, the first device may be turned off, the first device may be out of battery, the sensor coupled to the first device may fail, and the like. In a fail-safe mode, the second device may present an audio signal (e.g., trigger an alarm) at the latest time set by the user, even if data representing the certain sleep state is not received. For example, a latest time at which audio content is to be presented to wake a user up is received at the second device. Data representing a sleep state may be generated by a first device and transmitted to the second device. If the second device receives data representing a certain sleep state, such as light sleep, within a time period before the latest time, the second device may select and present the audio content at the time the data representing the certain sleep state is received. If the second device does not receive data representing the certain sleep state before the latest time, the second device may select and present the audio content at the latest time.

At 901, a first control signal comprising a latest time at which to receive a second control signal from a remote device to cause presentation of an audio signal is received. The second control signal may be, for example, generated by a remote device based on a sleep state determined by the remote device. The second control signal may be, for example, generated if and when a remote device detects a certain sleep state, such as light sleep. At 902, an inquiry may be made as to whether the current time is before the latest time. If no, the process goes to 904, and presentation of an audio signal comprising the audio content at a speaker is caused. Thus, the audio signal may be presented substantially at the latest time. If yes, the process goes to 903, and an inquiry may be made as to whether the second control signal is received from the remote device. If no, the process goes back to 902. The process may continue to wait for the second control signal to be received until the current time is passed the latest time. If yes, the process goes to 904, and presentation of an audio signal comprising the audio content at a speaker is caused. Thus, the audio signal may be presented substantially at the time the second control signal is received. Still, other processes may be possible.

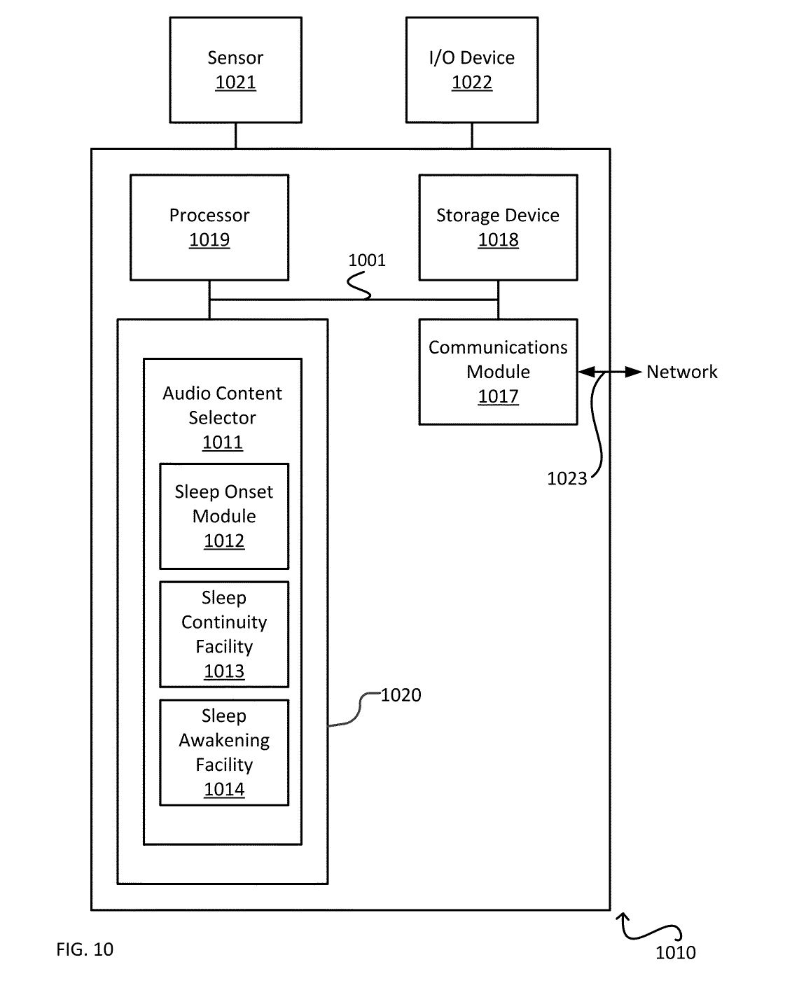

FIG. 10 illustrates a computer system suitable for use with a sleep state manager, according to some examples. In some examples, computing platform 1010 may be used to implement computer programs, applications, methods, processes, algorithms, or other software to perform the above-described techniques. Computing platform 1010 includes a bus 1001 or other communication mechanism for communicating information, which interconnects subsystems and devices, such as processor 1019, system memory 1020 (e.g., RAM, etc.), storage device 1018 (e.g., ROM, etc.), a communications module 1017 (e.g., an Ethernet or wireless controller, a Bluetooth controller, etc.) to facilitate communications via a port on communication link 1023 to communicate, for example, with a computing device, including mobile computing and/or communication devices with processors. Processor 1019 can be implemented with one or more central processing units (“CPUs”), such as those manufactured by Intel® Corporation, or one or more virtual processors, as well as any combination of CPUs and virtual processors. Computing platform 1010 exchanges data representing inputs and outputs via input-and-output devices 1022, including, but not limited to, keyboards, mice, audio inputs (e.g., speech-to-text devices), user interfaces, displays, monitors, cursors, touch-sensitive displays, LCD or LED displays, and other I/O-related devices. An interface is not limited to a touch-sensitive screen and can be any graphic user interface, any auditory interface, any haptic interface, any combination thereof, and the like. Computing platform 1010 may also receive sensor data from sensor 1021, including a heart rate sensor, an accelerometer, a GPS receiver, a GSR sensor, a bioimpedance sensor, and the like.

According to some examples, computing platform 1010 performs specific operations by processor 1019 executing one or more sequences of one or more instructions stored in system memory 1020, and computing platform 1010 can be implemented in a client-server arrangement, peer-to-peer arrangement, or as any mobile computing device, including smart phones and the like. Such instructions or data may be read into system memory 1020 from another computer readable medium, such as storage device 1018. In some examples, hard-wired circuitry may be used in place of or in combination with software instructions for implementation. Instructions may be embedded in software or firmware. The term “computer readable medium” refers to any tangible medium that participates in providing instructions to processor 1019 for execution. Such a medium may take many forms, including but not limited to, non-volatile media and volatile media. Non-volatile media includes, for example, optical or magnetic disks and the like. Volatile media includes dynamic memory, such as system memory 1020.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM, EPROM, FLASH-EPROM, any other memory chip or cartridge, or any other medium from which a computer can read. Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 1001 for transmitting a computer data signal.

In some examples, execution of the sequences of instructions may be performed by computing platform 1010. According to some examples, computing platform 1010 can be coupled by communication link 1023 (e.g., a wired network, such as LAN, PSTN, or any wireless network) to any other processor to perform the sequence of instructions in coordination with (or asynchronous to) one another. Computing platform 1010 may transmit and receive messages, data, and instructions, including program code (e.g., application code) through communication link 1023 and communication interface 1017. Received program code may be executed by processor 1019 as it is received, and/or stored in memory 1020 or other non-volatile storage for later execution.

In the example shown, system memory 1020 can include various modules that include executable instructions to implement functionalities described herein. In the example shown, system memory 1020 includes audio content selector 1011, which may include sleep onset module 1012, sleep continuity facility 1013, and sleep awakening facility 1014. An audio content library may be stored on storage device 1018 or another memory.

Although the foregoing examples have been described in some detail for purposes of clarity of understanding, the above-described inventive techniques are not limited to the details provided. There are many alternative ways of implementing the above-described invention techniques. The disclosed examples are illustrative and not restrictive.

What is claimed:

1. A method, comprising:
receiving data representing a sleep state;
selecting a portion of audio content from a plurality of portions of audio content as a function of the sleep state, the plurality of portions of audio content being stored in a memory; and
causing presentation of an audio signal comprising the portion of audio content at a speaker.

2. The method of claim 1, further comprising:
processing sensor data received from one or more sensors to determine a match with a sensor data pattern that includes a subset of data indicating a state of sleep preparation; and
identifying the data representing the sleep state as the state of sleep preparation.

3. The method of claim 1, further comprising:
receiving sensor data from one or more sensors;
comparing the sensor data to one or more sensor data patterns that include one or more subsets of data indicating one or more sub-sleep states; and
identifying the data representing the sleep state as one of the one or more sub-sleep states.

4. The method of claim 1, wherein the portion of audio content comprises a white noise configured to mask a background noise.

5. The method of claim 1, further comprising:
receiving another audio signal comprising a background noise,
wherein the portion of audio content is configured to substantially cancel the background noise.

6. The method of claim 1, wherein the causing presentation of the audio signal comprises causing presentation of a recommendation.

7. The method of claim 6, wherein the causing presentation of the recommendation comprises causing presentation of a recommendation to do an exercise.

8. The method of claim 1, wherein the causing presentation of the audio signal comprises causing presentation of a name of a user.

9. (canceled)

10. The method of claim 1, wherein the causing presentation of the audio signal comprises causing presentation of a news story.

11. The method of claim 1, wherein the causing presentation of the audio signal comprises causing presentation of a user's schedule.

12. The method of claim 1, further comprising:
receiving data representing an interference; and
causing an increase in a magnitude of the audio signal.

13. The method of claim 1, further comprising:
receiving data representing an interference; and
selecting the portion of audio content from the plurality of portions of audio content as a function of the sleep state and the interference.

14. The method of claim 13, wherein the interference comprises another audio signal comprising a snoring of a first user, and the portion of audio content is configured to substantially cancel the another audio signal received at a second user.

15. The method of claim 13, wherein the interference comprises another audio signal comprising a snoring of a user, and the portion of audio content comprises a name of the user.

16. The method of claim 1, further comprising:
selecting the portion of audio content from the plurality of portions of audio content as a function of a time period between the receiving the data representing the sleep state and the causing presentation of the audio signal comprising the portion of audio content at the speaker.

17. The method of claim 1, further comprising:
receiving a first control signal comprising a latest time at which to receive a second control signal from a remote device to cause presentation of the audio signal comprising the portion of audio content;
determining whether the second control signal is received from the remote device before the latest time; and
causing presentation of the audio signal comprising the portion of audio content substantially at a time when the second control is received if the second control signal is received from the remote device before the latest time, or causing presentation of the audio signal comprising the portion of audio content substantially at the latest time if the second control is not received from the remote device before the latest time.

18. The method of claim 1, further comprising:
receiving a control signal comprising a latest time at which to cause presentation of the audio signal comprising the portion of audio content;
determining a time period between a current time and the latest time; and
selecting the portion of audio content from the plurality of portions of audio content as a function of the time period.

19. The method of claim 1, further comprising:
determining that data representing another sleep state is not received within a time period after the causing presentation of the audio signal comprising the portion of audio content; and
causing presentation of another audio signal comprising another portion of audio content at the speaker.

20. A system, comprising:
a memory configured to store data representing a sleep state and to store a plurality of portions of audio content; and
a processor configured to select a portion of audio content from a plurality of portions of audio content as a function of the sleep state, and to cause presentation of an audio signal comprising the portion of audio content at a speaker.

21. The method of claim 3, wherein the one or more sub-sleep states comprise light sleep, deep sleep, and REM sleep.
